(19) United States
(12) Patent Application Publication
(10) Pub. No.: US 2016/0320325 A1
(43) Pub. Date: Nov. 3, 2016

(54) DEEP-FRYER OIL MEASURING APPARATUS

(71) Applicant: Testo AG, Lenzkirch (DE)

(72) Inventor: Andreas Derr, Donaueschingen (DE)

(73) Assignee: Testo AG, Lenzkirch (DE)

(21) Appl. No.: 15/139,675

(22) Filed: Apr. 27, 2016

(30) Foreign Application Priority Data
Apr. 28, 2015 (CN) 201520507430.2

(51) Int. Cl. G01N 27/22; G01N 33/03; A47J 37/12

(52) U.S. Cl. CPC G01N 27/221; A47J 37/1271; G01N 33/03

(57) ABSTRACT

A deep-fryer oil measuring apparatus is provided having a tubular probe (2), an oil sensor (4) which is disposed on a distal end of the tubular probe (2), and a handle (3) which is disposed on a proximal end of the tubular probe (2). Here, the handle (3) is disposed so as to be angular to the tubular probe (2).

### INCORPORATION BY REFERENCE

The following documents are incorporated herein by reference as if fully set forth: Chinese Patent Application No. 201520507430.2, filed Apr. 28, 2015.

### BACKGROUND

The invention relates to a deep-fryer oil measuring apparatus having a tubular probe, an oil sensor which is disposed on a distal end of the tubular probe, and a handle which is disposed on a proximal end of the tubular probe.

In order to determine the quality of deep-fryer oil which in particular has already been used for deep frying, the “total polar materials” (TPM), which comprise free fatty acids, monoglycerides and diglycerides, are conventionally measured in the oil. The latter are a measure of the thermo-oxidative decomposition of fat and represent a usually used unit of measure for stating the quality of deep-fryer oil, it fundamentally applying here that there is more decomposition the longer the oil is employed. The TPM value may be determined by column chromatography, for example. Furthermore, deep-fryer oil measuring apparatuses of the type mentioned at the outset, which are adapted for determining the percentage TPM value by in particular capacitive measuring by means of an oil sensor on a distal end of a tubular probe, which is held into the deep-fryer oil, are known.

Previously known deep-fryer oil measuring apparatuses have the disadvantage that they are to some extent complex in terms of handling and are thus uncomfortable to use for the operator. In particular, it may be only possible in a very complex manner for the operator to read a measured value from a display unit which is disposed on the handle, for example, since said display unit is typically disposed on the longitudinal side in relation to the tubular probe. Therefore, the operator cannot look directly from above onto the display unit which is configured on the handle, in particular not while the oil probe is dipped into a bath of deep-fryer oil. Furthermore, the storage capability of previously known deep-fryer oil measuring apparatuses is worthy of improvement.

### SUMMARY

There is, therefore, the object of providing a deep-fryer oil measuring apparatus of the type mentioned at the outset, which in particular overcomes the disadvantages mentioned above.

The object underlying this invention can be achieved by a deep-fryer oil measuring apparatus with one or more features of the invention. In order for the object to be achieved, it is in particular provided according to the invention in the deep-fryer oil measuring apparatus described at the outset that the handle is disposed so as to be angular to the tubular probe. By way of the angular arrangement of the handle in relation to the tubular probe it is possible for the first time for a user of the deep-fryer oil measuring apparatus according to the invention to be able to read a measured value in particular from a display unit which is preferably on the handle in a simpler and more comfortable manner, for example, in particular while said user simultaneously carries out a measurement by means of the oil sensor which is dipped into the deep-fryer oil, and/or for said user to be able to operate the deep-fryer oil measuring apparatus according to the invention in a simpler and more comfortable manner, in particular without having to take the deep-fryer oil measuring apparatus according to the invention out of the deep-fryer oil, and/or having to change the view angle in relation to the deep-fryer oil measuring apparatus according to the invention. The oil sensor may serve for measuring the quality of the deep-fryer oil and/or the temperature of the deep-fryer oil, for example.

It is often the case that previously known deep-fryer oil measuring apparatuses are damaged by inappropriate application, in particular by the employment of the former as a lever, above all by inappropriate exertion of pressure on the oil sensor. A further advantage of the deep-fryer oil measuring apparatus according to the invention thus lies in that inappropriate use of the deep-fryer oil measuring apparatus according to the invention as a lever, for example, is avoided by the handle which is disposed so as to be angular in relation to the tubular probe, since as opposed to previously known deep-fryer oil measuring apparatuses, the deep-fryer oil measuring apparatus according to the invention, by virtue of the construction concept thereof, is unsuitable therefor.

In particular, it may be provided that the deep-fryer oil measuring apparatus according to the invention has a capacitive oil sensor.

In order to guarantee handling of the deep-fryer oil measuring apparatus according to the invention that is as comfortable and simple as possible, it may be provided that the handle in relation to the tubular probe encloses an angle of at most 110° and/or at the least 80°. The handle in relation to the tubular probe may preferably enclose a right angle. Due to this, the user may read a measured value from a display device, for example, while the deep-fryer oil measuring apparatus according to the invention is dipped into the deep-fryer oil.

An ergonomically advantageous arrangement according to the invention may provide that the handle is disposed so as to be laterally offset in relation to a central axis of the tubular probe. On account thereof, handling of the deep-fryer oil measuring apparatus according to the invention by the user may be improved even further.

In order for the user of the deep-fryer oil measuring apparatus according to the invention to be able to grip and/or hold the handle as securely as possible, it may be expedient for the deep-fryer oil measuring apparatus to have a finger depression. The finger depression may be provided in particular on a distal side of the handle.

It may be furthermore provided that the deep-fryer oil measuring apparatus according to the invention at least at one end of the at least one finger depression has a limiting bracket which is preferably curved towards the at least one finger depression. This enables a yet even more improved grip and/or protection of the hand holding the handle.

In order to achieve a construction concept of the deep-fryer oil measuring apparatus according to the invention that is as compact and space-saving as possible, it may be provided that an evaluation unit for the oil sensor is disposed in the handle. The evaluation unit serves for detecting and/or evaluating one or a plurality of measured values.

In one further advantageous design embodiment of the invention that may potentially be of independent inventive quality in particular in a deep-fryer oil measuring apparatus according to the invention having a tubular probe, a capacitive oil sensor which is disposed on a distal end of the tubular probe, and a handle which is disposed on a proximal end of the tubular probe, it may be provided that the tubular probe is releasably fastened to the handle. This enables, for example, that the user may store a or the tubular probe and the handle in a mutually independent and in particular space-saving manner. Furthermore, this design embodiment according to the invention has the advantage that an or the oil sensor which is disposed on a distal end of a or the tubular probe may be cleaned independently, in particular without the handle, together with the tubular probe.

It may furthermore be expedient for a or the tubular probe having the or an evaluation unit for the oil sensor to be removably disposed on the handle.

In order to be able to reversibly and/or stably connect a or the tubular sensor to the handle, it may be provided in the deep-fryer oil measuring apparatus according to the invention that a or the tubular probe in the use position is held on the handle by a screw connection. It may be provided in particular that a or the tubular probe is fastenable to the handle by a lock nut, preferably by screwing into a thread.

A particularly expedient and in particular cost-effective and/or space-saving design embodiment of the deep-fryer oil measuring apparatus according to the invention may provide that an or the evaluation unit is disposed on a or the tubular probe in an in particular elongate cartridge which is longitudinally aligned with the tubular probe. A further advantage of the invention lies in that, on account thereof, for calibrating/gauging in particular of the evaluation unit more tubular probes without a handle are insertable into an oil bath having known parameters than if an (obliquely placed) handle would remain on a tubular probe. This enables that significantly more tubular probes, in particular having the evaluation units disposed in the tubular probes, may be simultaneously calibrated/gauged during a single calibration procedure, due to which time may be saved during calibration.

It may furthermore be expedient for the handle to have a clearance having an internal contour which is configured so as to match an external contour of the cartridge. This enables a space-saving arrangement of the individual components and/or a stable connection between the individual components of the deep-fryer oil measuring apparatus according the invention.

In order for the cartridge to be able to be adequately ventilated, it may be provided that the cartridge has ventilation slots.

It may be provided that a display unit is configured on the handle. In particular, it may be provided that a or the display unit is configured on a proximal side of the handle.

It may furthermore be provided that a or the display unit is disposed in an extension of the tubular probe. The display unit is thus easily readable using an outstretched arm, for example, and/or it is more readily checkable during reading whether the tubular probe is placed at a correct measuring point.

In order to be able to read measured values in a comfortable and simple manner, for example, it may be provided that a or the display unit defines a preferred reading direction which in particular is longitudinally aligned with the tubular probe.

In particular, it may be provided that a or the display unit has a preferably graphics-capable liquid crystal display.

Since it may be particularly provided that as opposed to previously known deep-fryer oil measuring apparatuses, the display unit of the deep-fryer oil measuring apparatus according to the invention is not longitudinally aligned with the tubular probe, the user in a simple and comfortable manner may direct his/her view toward the display unit, without having to change his/her angle of view onto the display unit and/or having to remove the deep-fryer oil measuring apparatus from the deep fryer.

One advantageous embodiment of the invention, which offers particularly high operator comfort, may provide that one or a plurality of operating elements for the or a display unit is/are configured on the handle. A further advantageous embodiment of the invention may provide that one or a plurality of operating elements for the or an evaluation unit of the oil sensor is/are configured on the handle. In particular, it may be expedient for the operating element or the operating elements to be configured on the or a proximal side of the handle, and/or so as to be level with the at least one or at least one finger depression, such that the user may reach said operating elements as easily as possible, in particular with his/her hand holding the deep-fryer oil measuring apparatus according to the invention, in particular without having to use his/her hand not holding the deep-fryer oil measuring apparatus according to the invention, in order to actuate the operating element or the operating elements.

In the context of the invention, the term distal means a side of a part of the deep-fryer oil measuring apparatus according to the invention that faces away from the body of the user.

In the context of the invention, the term proximal means a side of a part of the deep-fryer oil measuring apparatus according to the invention that faces the body of the user.

### BRIEF DESCRIPTION OF THE DRAWINGS

A specific embodiment of the invention is described in more detail hereunder by the drawings. In a schematic illustration:

FIG. 1 shows a perspective view of a specific embodiment of the deep-fryer oil measuring apparatus according to the invention;

FIG. 2 shows a perspective view of the specific embodiment of the deep-fryer oil measuring apparatus according to the invention of FIG. 1, wherein the tubular probe in the illustration is present so as to be separate from the handle; and

FIG. 3 shows an exploded drawing of a tubular probe of the deep-fryer oil measuring apparatus according to the invention.

### DETAILED DESCRIPTION OF THE PREFERRED EMBODIMENTS

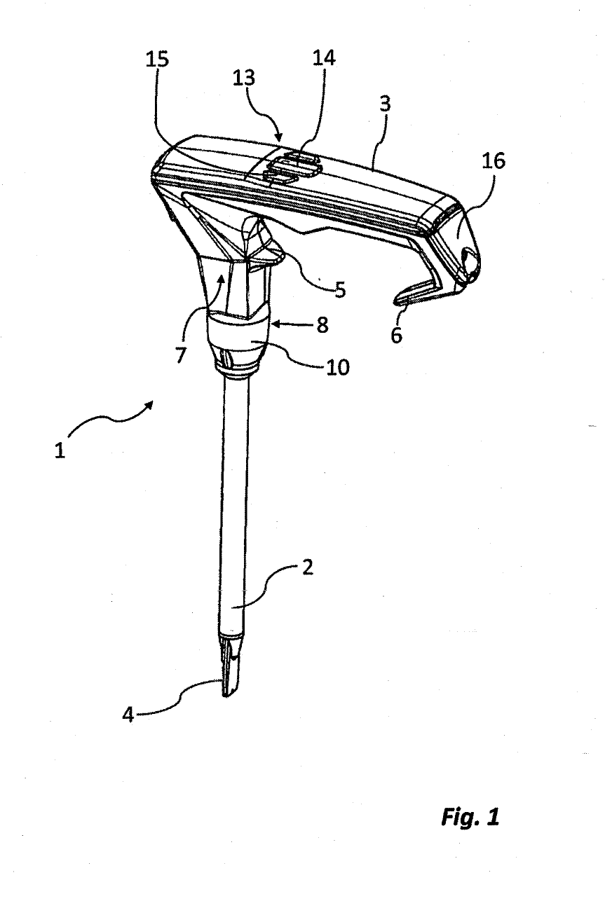

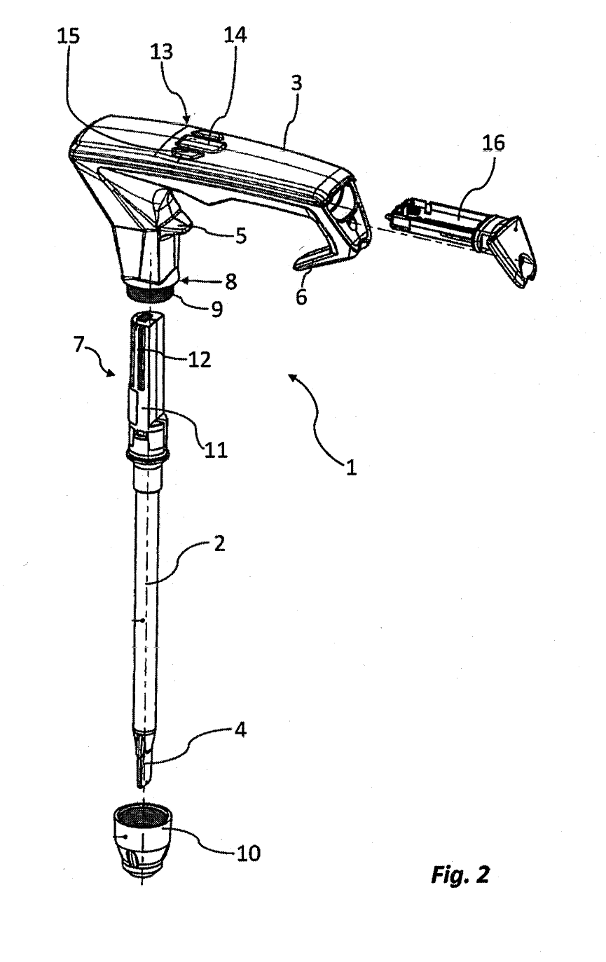

In FIGS. 1 and 2 a deep-fryer oil measuring apparatus according to the invention, which in its entirety is denoted by 1, is depicted in a perspective illustration.

The deep-fryer oil measuring apparatus 1 according to the invention has a tubular probe 2 having an in particular capacitive oil sensor 4 which is disposed on a distal end of the tubular probe 2, and a handle 3 which is disposed on a proximal end of the tubular probe 2. The deep-fryer oil measuring apparatus 1 according to the invention is configured in such a manner that the tubular probe 2 is releasably fastened to the handle 3. The oil sensor 4 serves for measuring the temperature and/or for measuring the polar materials, by way of which in particular the measured value of total polar materials (TPM) in the oil that are contained in the deep-fryer oil may be determined. Measuring the values of the deep-fryer oil by means of the deep-fryer oil measuring apparatus according to the invention is capable of being carried out in a simple and comfortable manner, even while the deep-fryer oil is located in the deep fryer.

In order for measuring to be carried out in a simplified and more comfortable manner, preferably in a deep fryer, it is provided that the handle 3 is disposed so as to be angular to the tubular probe 2. In the illustrated embodiment of the invention the handle 3 is disposed so as to be at an approximately right angle to the tubular probe 2, and so as to be laterally offset in relation to a central axis of the tubular probe 2.

A further advantage of the deep-fryer oil measuring apparatus 1 according to the invention lies in that the tubular probes 2 which are configured to be removable, may be stored in a more space-saving manner and independently from the handle.

The handle 3 has a finger depression 5 which is disposed on the distal side of the handle 3, a limiting bracket 6 being configured at an end of the latter, which runs in a manner curved towards the finger depression 5. This guarantees that the user during operation can securely hold the deep-fryer oil measuring apparatus 1 according to the invention and/or does not unintentionally lose his/her grip thereon. The embodiment according to the invention of the deep-fryer oil measuring apparatus 1 furthermore has the advantage that handling is possible using just one hand.

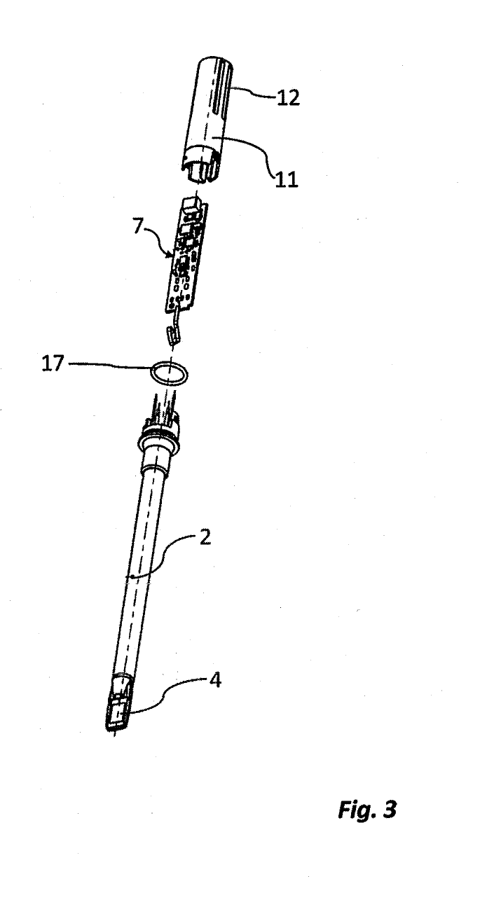

The deep-fryer oil measuring apparatus 1 according to the invention has an evaluation unit 7 for the oil sensor 4, the former being disposed on the tubular probe 2 in an elongate cartridge 11 which is longitudinally aligned with the tubular probe 2 as shown in FIGS. 2 and 3. Together with the tubular probe 2, the evaluation unit 7 for the oil sensor 4, which is disposed in the cartridge 11, is disposed so as to extractable on the handle 3, wherein a ring-shaped annular seal 17 is disposed between the cartridge 11 and the tubular probe 2 for sealing.

As is shown in FIG. 2, the cartridge is disposed on a free end of the tubular probe 2 that lies opposite the distal free end of the tubular probe 2 that comprises the oil sensor 4. In this way, the cartridge 11 may be inserted completely into a clearance having an internal contour that is moulded on the handle 3 and that is configured so as to match an external contour of the cartridge 11. Due to this, a comparatively compact and advantageous construction concept results, for example on account thereof, that the cartridge 11 is present disposed within the handle 3 so as to be protected from shocks in the use position.

The evaluation unit 7 in the case of previously known deep-fryer oil measuring apparatuses is usually fixedly integrated within a housing of a handle, for example. By way of the removable tubular probe 2 and of the evaluation unit 7 which is disposed in the cartridge 11 on the tubular probe 2, an advantageous and synergetic effect results such that an evaluation unit 7 for the first time is capable of being calibrated independently of the handle 3. Moreover, the arrangement according to the invention of the deep-fryer oil measuring apparatus 1 has the advantage that, due to this construction, for calibrating the evaluation unit 7 more tubular probes 2 without a handle 3 are insertable into an oil bath having known parameters, as to when a handle 3 would remain on the tubular probe 2. This enables significantly more evaluation units 7 disposed in the tubular probes 2 to be calibrated during a single calibration procedure, since only a limited number of deep-fryer oil measuring apparatuses 1 typically simultaneously find space in one oil bath having known parameters. Without a handle 3 which is connected to the tubular probe 2, individual tubular probes 2 may simultaneously be employed in the oil bath in a more densely packed manner during calibrating, due to which savings in time and/or in space are possible during calibrating.

In order for the tubular probe 2 to be able to be fixedly connected to the handle 3, the deep-fryer oil measuring apparatus 1 according to the invention has a screw connection 8. In the embodiment of the invention which is illustrated in FIGS. 1 and 2, the handle 3 has a thread 9 which is moulded as an external thread onto which a lock nut 10, which has a counter thread matching the thread 9, is screw-fittable. By screwing the lock nut 10 thereon, a clamping force which fixedly holds together the tubular probe 2 and the handle 3 in a connected state may be generated. The tubular probe 2 is thus configured so as to be extractable together with the evaluation unit 7 from the handle 3, and may be separated from the handle 3 when the lock nut 10 is not present so as to have been screw-fitted onto the thread 9.

A plurality of ventilation slots 12, which are longitudinally aligned with the tubular probe 2, are configured on the cartridge 11, in order to improve ventilation of the evaluation unit 7.

In order for the user of the deep-fryer oil measuring apparatus 1 according to the invention to be able to read the measured values of the deep-fryer oil in a simple and comfortable manner, the deep-fryer oil measuring apparatus 1 according to the invention has a display unit 13 which is configured on the proximal side of the handle 3 and which has a graphics-capable liquid crystal display 14. The display unit 13 here defines a preferred reading direction which is longitudinally aligned with the tubular probe 2.

In order for the deep-fryer oil measuring apparatus 1 according to the invention to be able to be operated in a simple and comfortable manner, in particular using only one hand, the handle 3 has operating elements 15 for the display unit 13 and/or for the evaluation unit 7 of the oil sensor. In the specific embodiment of the invention according to FIGS. 1 and 2 the operating elements 15 are configured on a proximal side of the handle 3. Alternatively or additionally, the operating elements 15 may also be disposed so as to be level with the finger depression 5.

The handle 3 furthermore has an extractable battery compartment 16 into which batteries and/or rechargeable power packs for the power supply of the deep-fryer oil measuring apparatus 1 according to the invention are insertable.

### LIST OF REFERENCE SIGNS

1 Deep-fryer oil measuring apparatus

2 Tubular probe

3 Handle

4 Oil sensor

5 Finger depression

6 Limiting bracket

7 Evaluation unit

8 Screw connection

9 Thread

10 Lock nut

11 Cartridge

12 Ventilation slots

13 Display unit

14 Liquid crystal display

15 Operating element

16 Battery compartment

17 Annular seal

1. A deep-fryer oil measuring apparatus (1), comprising: a tubular probe (2), an oil sensor (4) disposed on a distal end of the tubular probe (2), and a handle (3) disposed on a proximal end of the tubular probe (2), the handle (3) is arranged at an angle to the tubular probe (2).

2. The deep-fryer oil measuring apparatus (1) according to claim 1, wherein the angle between the handle (3) and the tubular probe (2) is at most 110° or at least 80°.

3. The deep-fryer oil measuring apparatus (1) according to claim 1, wherein the handle (3) is disposed laterally offset in relation to a central axis of the tubular probe (2).

4. The deep-fryer oil measuring apparatus (1) according to claim 1, wherein the handle (3) further comprises at least one finger depression (5) located on a distal side of the handle (3).

5. The deep-fryer oil measuring apparatus (1) according to claim 4, further comprising a limiting bracket (6) at least at one end of the at least one finger depression (5).

6. The deep-fryer oil measuring apparatus (1) according to claim 1, further comprising an evaluation unit (7) for the oil sensor (4) disposed in the handle (3).

7. The deep-fryer oil measuring apparatus (1), comprising: a tubular probe (2), a capacitive oil sensor (4) disposed on a distal end of the tubular probe (2), and a handle (3) disposed on a proximal end of the tubular probe (2), with the tubular probe (2) being releasably fastened to the handle (3).

8. The deep-fryer oil measuring apparatus (1) according to claim 7, wherein the tubular probe (2) includes an evaluation unit (7) for the oil sensor (4) removably disposed on the handle (3).

9. The deep-fryer oil measuring apparatus (1) according to claim 7, wherein the tubular prove (2) is held on the handle (3) by a screw connection (8, 9, 10).

10. The deep-fryer oil measuring apparatus (1) according to claim 8, wherein the evaluation unit (7) is disposed on the tubular probe (2) in a cartridge (11) which is longitudinally aligned with the tubular probe (2).

11. The deep-fryer oil measuring apparatus (1) according to claim 10, wherein the handle (3) has a clearance having an internal contour which is configured to match an external contour of the cartridge (11).

12. The deep-fryer oil measuring apparatus (1) according to claim 10, wherein the cartridge (11) has ventilation slots (12).

13. The deep-fryer oil measuring apparatus (1) according to claim 8, further comprising a display unit (13) is configured on the handle (3).

14. The deep-fryer oil measuring apparatus (1) according to claim 13, wherein the display unit (13) is disposed in an extension of the tubular probe (2).

15. The deep-fryer oil measuring apparatus (1) according to claim 13, wherein the display unit (13) defines a preferred reading direction longitudinally aligned with the tubular probe (2).

16. The deep-fryer oil measuring apparatus (1) according to claim 13, wherein the display unit (13) comprises a liquid crystal display (14).

17. The deep-fryer oil measuring apparatus (1) according to claim 13, further comprising operating elements (15) for the display unit (13) or for the evaluation unit (7) of the oil sensor (4), or both, are configured on the handle (3),

18. The deep-fryer oil measuring apparatus (1) according to claim 17, wherein the operating elements (15) are configured to be level with at least one finger depression (5) located in the handle (3).

19. The deep-fryer oil measuring apparatus (1) according to claim 1, wherein the angle between the handle (3) and the tubular probe (2) is at most 110° and at least 80°.

20. The deep-fryer oil measuring apparatus (1) according to claim 1, wherein the deep-fryer oil measuring apparatus has a capacitive oil sensor (4).

21. The deep-fryer oil measuring apparatus (1) according to claim 5, wherein the finger limiting bracket (6) is curved towards the at least one finger depression (5).
